(19) United States
(12) Patent Application Publication
(10) Pub. No.: US 2009/0232831 A1
(43) Pub. Date: Sep. 17, 2009

(54) METHODS AND COMPOSITIONS FOR THE TREATMENT OR PREVENTION OF HUMAN IMMUNODEFICIENCY VIRUS INFECTION

(76) Inventors: Chi-Huey Wong, La Jolla, CA (US); Sheng-Kai Wang, Taipei (TW); Pi-Hui Liang, Taipei (TW)

Correspondence Address: Luce, Forward, Hamilton & Scripps LLP, 2050 Main Street, Suite 600, Irvine, CA, 92614, US

(21) Appl. No.: 12/338,987

(22) Filed: Dec. 18, 2008

Related U.S. Application Data
(60) Provisional application No. 61/036,432, filed on Mar. 13, 2008.

(51) Int. Cl. A61K 39/42; A61K 31/715; C07H 1/00; C12Q 1/70

(52) U.S. Cl. 424/160.1; 514/54; 536/123.1; 435/5

(57) ABSTRACT

A conserved cluster of oligomannose glycans on gp120 has been identified as the epitope recognized by the broadly HIV-1-neutralizing monoclonal antibody 2G12. Oligomannose glycans are also the ligands for DC-SIGN, a C-type lectin found on the surface of dendritic cells. Multivalency is fundamental for carbohydrate-protein interactions, and mimicking of the high glycan density on the virus surface has become essential for designing carbohydrate-based HIV vaccines and antiviral agents. Synthesis of oligomannose dendrons, which display multivalent oligomannoses in high density, and characterize their interaction with 2G12 and DC-SIGN by a glycan microarray binding assay is disclosed. These glycodendrons inhibit the binding of gp120 to 2G12 and recombinant dimeric DC-SIGN with IC₅₀ in the nanomolar range. A second-generation Man₉ dendron was identified as a potential immunogen for HIV vaccine development and as a potential antiviral agent.

### RELATED APPLICATIONS

This nonprovisional patent application claims priority to U.S. Provisional Patent Application Ser. No. 61/049,358 filed on Apr. 30, 2008.

### BACKGROUND

This disclosure relates to novel compositions, methods, and kits for addressing HIV infection, as well as preventing HIV infection. Moreover, the compositions and methods of the present disclosure provide a novel platform for research related to prevention and treatment of HIV.

### SUMMARY

It is widely accepted that the heavily glycosylated glycoprotein gp120 on the surface of HIV-1 shields peptide epitopes from recognition by the immune system and may promote infection in vivo by interaction with dendritic cells and transport to tissue rich in CD4 T cells. A conserved cluster of oligomannose glycans on gp120 has been identified as the epitope recognized by the broadly HIV-1-neutralizing monoclonal antibody 2G12. Oligomannose glycans are also the ligands for DC-SIGN, a C-type lectin found on the surface of dendritic cells. Multivalency is fundamental for carbohydrate-protein interactions, and mimicking of the high glycan density on the virus surface has become essential for designing carbohydrate-based HIV vaccines and antiviral agents. An efficient synthesis of oligomannose dendrons, which display multivalent oligomannoses in high density, and characterize their interaction with 2G12 and DC-SIGN by a glycan microarray binding assay is disclosed. The solution and the surface binding analysis of 2G12 to a prototype oligomannose dendron clearly demonstrated the efficacy of dendrimeric display. These glycodendrons inhibit the binding of gp120 to 2G12 and recombinant dimeric DC-SIGN with IC₅₀ in the nanomolar range. A second-generation Man₉ dendron was identified as a potential immunogen for HIV vaccine development and as a potential antiviral agent.

According to a feature of the present disclosure, a method is disclosed comprising addressing the infection of a human immunodeficiency virus (HIV) by administering a composition comprising oligomannose dendrons to a patient to induce production of antibodies that will recognize the HIV or compete with the HIV for DC-SIGN binding.

According to a feature of the present disclosure, a composition is disclosed comprising a oligomannose dendrimer and a pharmaceutically acceptable carrier.

According to a feature of the present disclosure, a composition is disclosed comprising a vaccine to address a human immunodeficiency virus infection comprising at least an oligomannose dendrimer.

According to a feature of the present disclosure, a method is disclosed comprising administering a composition to a patient at risk for acquiring a human immunodeficiency virus infection, the composition comprising an oligomannose dendrimer and a pharmaceutically acceptable carrier.

According to a feature of the present disclosure, a method is disclosed comprising manufacturing an oligomannose dendrimer have the steps shown in at least one of FIG. 4 or FIG. 5.

According to a feature of the present disclosure, a method is disclosed comprising screening at least one antibody for activity for efficacy against a human immunodeficiency virus by contacting the at least one antibody with a substrate having bound thereto oligomannose dendrimers, and detecting the presence or absence of a probe.

### DRAWINGS

The above-mentioned features and objects of the present disclosure will become more apparent with reference to the following description taken in conjunction with the accompanying drawings wherein like reference numerals denote like elements and in which:

FIG. 1 are implementations of structures of Man₉(GlcNAc)₂ and synthetic Man₄ and Man₉ according to the present disclosure;

FIG. 2 is an illustration implementations of two strategies for targeting HIV-1 by oligomannose dendrons;

FIG. 3 are illustrations of implementations of monomeric and multivalent oligomannose binding to 2G12 complexes;

FIG. 4 is a schematic of an implementation of syntheses of first-, second-, and third-generation alkynyl dendrimeric scaffolds, where EDC is 1-(3-dimethyl-aminopropyl)-3-ethylcarbodiimide hydrochloride; DIEA is diisopropylethylamine; and DMF is dimethylformamide;

FIG. 5 is a schematic of an implementation of conjugation of oligomannose to alkynyl scaffolds by CuAAC reaction;

FIG. 6 a graph of an implementation of experimental data representative of MALDI-TOF mass spectra for a glycodendron of the present disclosure;

FIGS. 7A-7C are an illustration and implementations of experimental data showing measurement of oligomannose dendrons-2G12 complex interaction by glycan array competition assay;

FIGS. 8A-8B are implementations of experimental data showing the properties of glycodendron 17-coated slides;

FIGS. 9A-9D are implementations of flow cytometry data showing oligomannose dendrons bind cell-surface receptors;

FIG. 10A is a MALDI-TOF Mass Spectrum for compound 15;

FIG. 10B is a MALDI-TOF Mass Spectrum for compound 17;

FIG. 11A is a graph of implementations of experimental data showing inhibition of 2G12 binding to normal density Man₄ slide at various concentrations of different glycodendrons;

FIG. 11B is a graph of implementations of experimental data showing inhibition of 2G12 binding to high density Man₄ slide at various concentrations of different glycodendrons; and

FIG. 12 is a graph of implementations of experimental data showing flow cytometric analysis of DC-SIGN expression.

### DETAILED DESCRIPTION

In the following detailed description of implementations of the invention, reference is made to the accompanying drawings in which like references indicate similar elements, and in which is shown by way of illustration specific implementations in which the invention may be practiced. These implementations are described in sufficient detail to enable those skilled in the art to practice the invention, and it is to be understood that other implementations may be utilized and that logical, mechanical, biological, electrical, functional, and other changes may be made without departing from the scope of the present invention. The following detailed description is, therefore, not to be taken in a limiting sense, and the scope of the present invention is defined only by the appended claims. As used in the present disclosure, the term “or” shall be understood to be defined as a logical disjunction and shall not indicate an exclusive disjunction unless expressly indicated as such or notated as “xor.”

This applications incorporates by reference U.S. Provisional Patent Application Ser. No. 61/049,358 filed on Apr. 30, 2008.

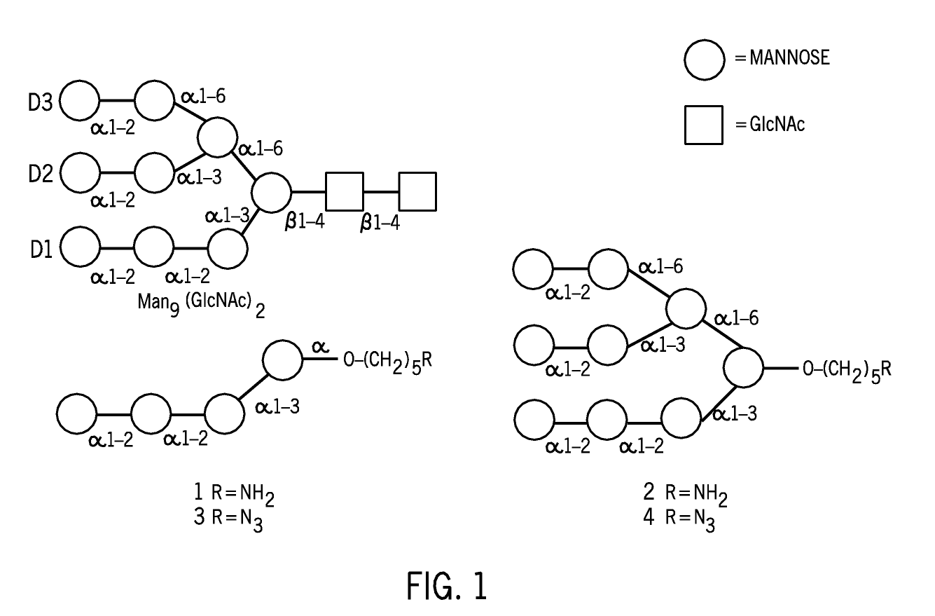

HIV infection is a massive global health problem with more than 33 million infected worldwide. An interesting feature of HIV is its densely glycosylated surface; the glycans account for 50% mass of the virus coat protein gp120. This carbohydrate face of gp120 aids in immune evasion and has been implicated in the enhancement of viral dissemination. Although the viral glycans are assembled by the host, their dense arrangements are relatively unique and the glycan shield has become an attractive potential target for the design of antiHIV-1 agents including vaccine-induced antibodies. However, all efforts directed toward antibody-based vaccine development so far have failed. Recently, a broad type-1 HIV neutralizing antibody, 2G12, was confirmed to recognize multiple high mannose glycans on gp120, suggesting that these glycans may be used for the design of an HIV vaccine component to elicit “2G12-like” antibodies. A combination of crystallographic, biochemical, and modeling studies has shown that two Man₉(GlcNAc)₂ (as illustrated according to implementations in FIG. 1), at positions 332 and 392, predominantly contribute to the gp120-2G12 interaction, while an oligomannose at position 339 may also contribute to the interaction.

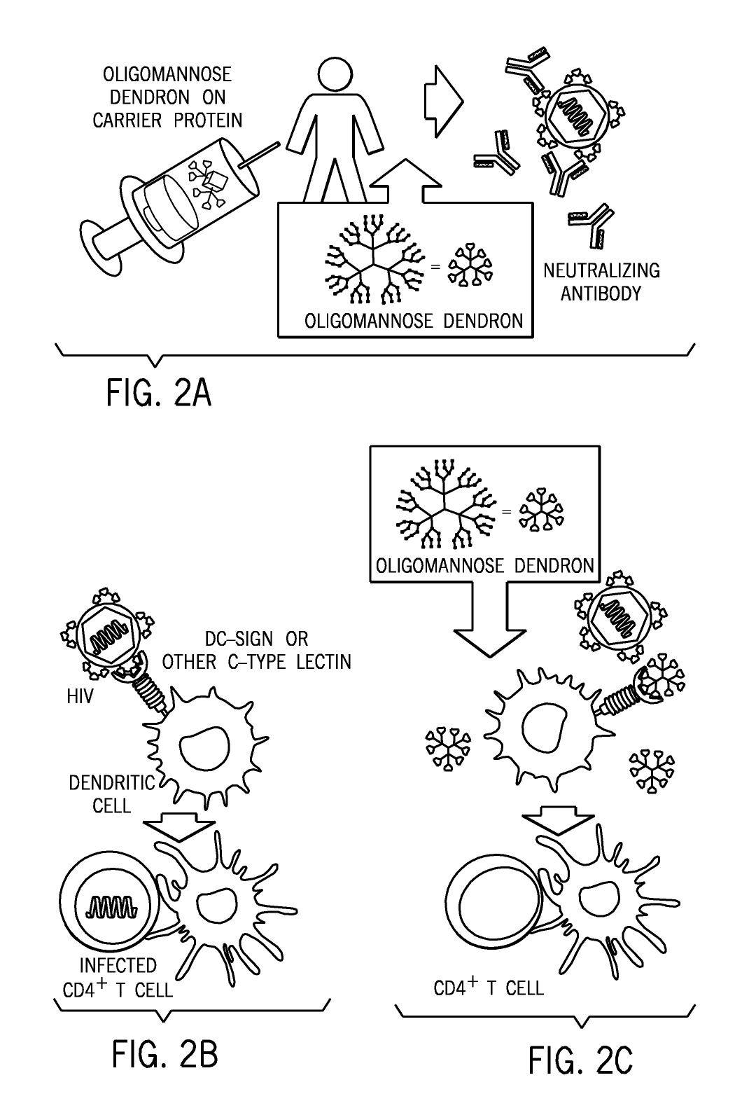

Another approach to anti-HIV activity is to block the interaction between HIV-1 and dendritic cells, which are associated with the enhanced infection of CD4 T cells (FIG. 2B). It has been proposed that the mannose-binding C-type lectin DC-SIGN (dendritic cell-specific intercellular adhesion molecule-grabbing nonintegrin) on dendritic cells interacts with the high-mannose glycans on HIV-1 and facilitates its dissemination, likely through the trans and cis mechanisms. This proposed mechanism is supported by studies that show that DC-SIGN binds the a1-43 and a1-46 mannotriose fragments. Therefore, as illustrated in FIG. 2, mimics of the multivalent N-linked high-mannose arrangement on gp120 have potential in HIV vaccine development and in the development of prophylactic antiviral agents that inhibit dendritic cell-mediated HIV-1 infection.

According to implementations shown in FIG. 2, glycodendrons are conjugated to carrier proteins and serve as vaccines in FIG. 2A. Thus, the compositions of the present disclosure address HIV prospectively, prior to infection. However, the compositions of the present disclosure are also useful after HIV infection. Accordingly and as illustrated in FIG. 2B, HIV-1 has been shown to bind dendritic cell-surface DC-SIGN or other mannose-binding proteins to enhance CD4 T cell infection. Thus, oligomannose dendrons can be injected to inhibit the binding of HIV viruses to dendritic cell-surface DC-SIGN or other mannose-binding proteins, thereby preventing dendritic cell-enhanced CD4 T cell infection.

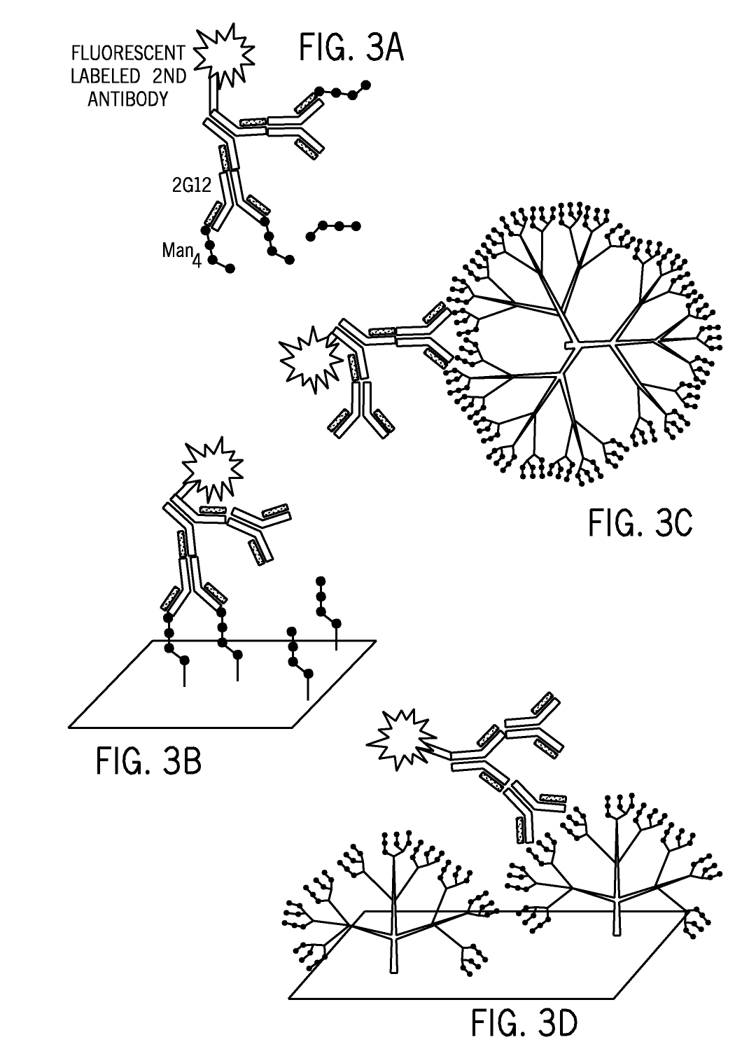

Oligomannoses corresponding to the D3 or D1 arm of Man₉(GlcNAc)₂ can mimic the complete glycan in disrupting the gp120-2G12 interaction. However, monomeric oligo-saccharides bind to 2G12 weakly, as illustrated in FIG. 3A. FIG. 3A-3D illustrates four modes of mannose binding by 2G12. In FIG. 3A, monomeric Man₄ in able to bind 2G12 in solution. However, 2G12's binding efficiency is greatest when it binds in a multivalent manner, as illustrated in FIG. 3B. Consequently, oligomannose dendrimers are shown to effectively bind 2G12 because it allows for multivalent binding. Similarly, immobilized oligomannose molecules, such as Man₄ or Man₉, are able to effectively bind to 2G12 in configurations that allow for multivalent binding, such as those disclosed in incorporated by reference U.S. Provisional Ser. No. 61/049,358.

Likewise, among various monomeric oligomannoses, the highly branched Man₉(GlcNAc)₂ is more potent in binding DC-SIGN; however, the affinity is low. Because the importance of multivalency in carbohydrate-protein interactions is well established, our design of HIV vaccines or anti-HIV carbohydrates is based on multivalent presentation of Man₉(GlcNAc)₂ and related glycans.

It was demonstrated that the interaction of Man₄ to 2G12 is greatly improved when Man₄ is immobilized onto a glass slide, as illustrated in FIG. 3B. However, this surface-generated pseudomultivalency is not suitable for vaccine purposes. To achieve the multivalent display of oligomannose on a carrier protein, we turned our attention to branched scaffolds, as shown in FIG. 3C.

Enhancement of carbohydrate-protein interactions by multivalency has been reported using glycoclusters, glycodendrimers/glycodendrons, and glycopolymers. Among these architectural categories, glycodendrons are suitable because (i) a relatively large number of carbohydrates can be displayed with a high density, (ii) their valency and size can be easily adjusted, and (iii) they can be selectively functionalized for conjugation to other biomolecules. Over the past several years, many groups have found that glycodendrimers/glycodendrons exhibit strikingly enhanced affinity against target proteins. Disclosed herein is a synthetic approach and application of these macromolecules.

### Syntheses of Oligomannose Dendrons.

An AB3 type dendrimeric skeleton was chosen because of its high loading number that can be achieved in a few generations. A versatile ligation reaction was also needed to conjugate sterically demanding oligomannose to the dendrons. The copper(I) catalyzed alkyne-azide 1,3-dipolar cycloaddition reaction (CuAAC) for this conjugation was exploited.

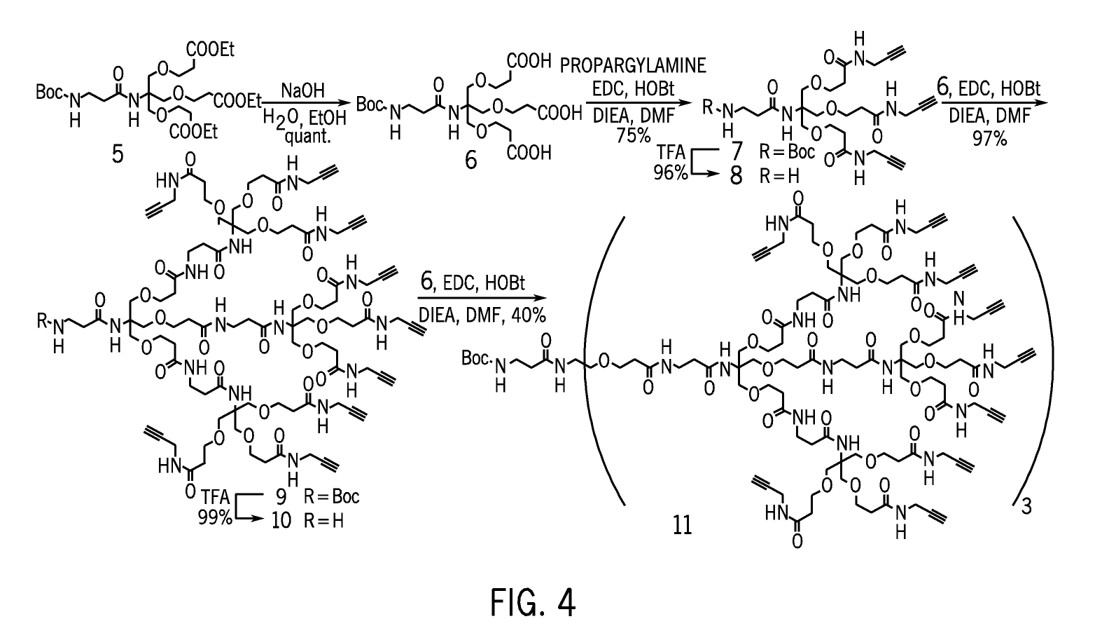

To facilitate homogeneity of the dendrimeric scaffold, the synthesis was designed by means of a convergent approach; terminal alkyne groups were installed on building block 6, as illustrated in FIG. 4, providing 7 as the first-generation tris-alkyne. Installing the alkyne at an early stage not only avoided incomplete alkynyl installation for later generation dendrons, but also saved a final deprotection step at the end of the synthesis. Tris-alkyne 7 was deprotected in trifluoroacetic acid solution to give 8, which was condensed to give a second generation alkynyl dendron 9. Using similar deprotection and condensation conditions, the third-generation alkynyl dendron 11 was synthesized.

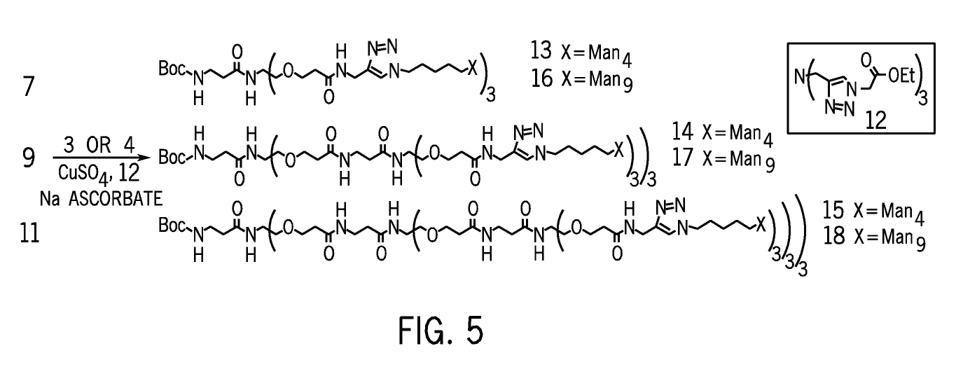

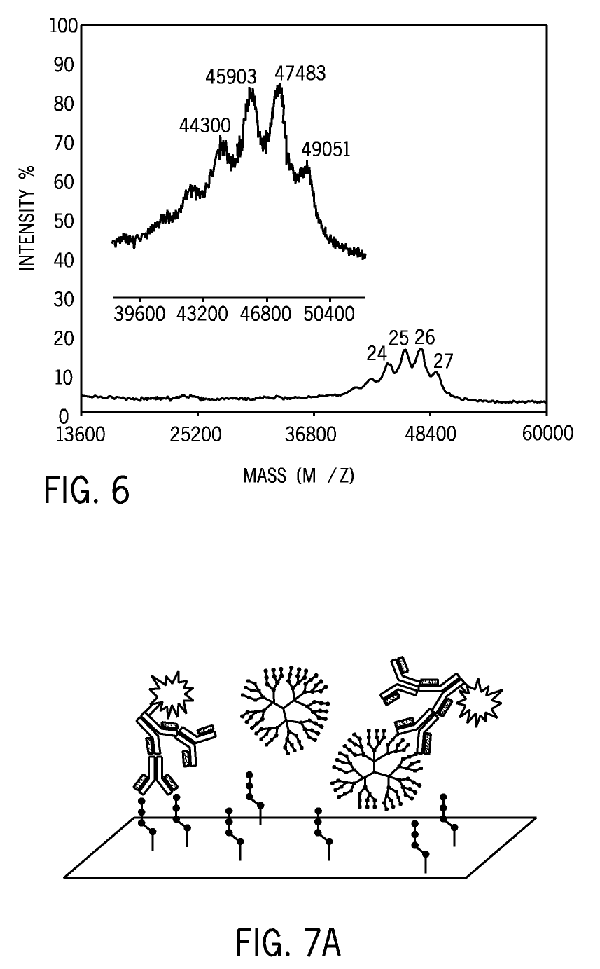

Next, azido-Man₄ or Man₉ (see FIG. 1) we coupled to different generations of alkynyl dendrons, as illustrated according to implementations shown in FIG. 5 via CuAAC. The reaction proceeded rapidly (<0.5 h) as monitored by matrix-assisted laser desorption/ionization-time-of-flight (MALDI-TOF) mass spectrometry, which also measured the conjugation copy number. It was found that with a longer reaction time and extra Cu^{I} complex, the reaction did not reach completion in the most sterically congested cases (i.e., 15, 17, and 18), as shown for example in FIG. 6. However, for compound 4, even for the third generation, a near-maximum occupancy was achieved. In comparison, using conventional amide coupling conjugation, only 8 of 32 sites were occupied by a hexasaccharide on a PAMAM dendrimer. The average numbers of attached oligomannose per dendron are summarized in Table 1.

TABLE 1
Average copy number of oligomannose dendrons, determined
by MALDI-TOF mass spectrometry

| | High mannose | | Expected | Average | |
|---|---|---|---|---|---|
| | Dendron type | | copy | copy | |
| Compound | generation | | number | number | Yield, % |
| 3 | Man4-N3 | | 1 | 1 | — |
| 13 | Man4 | 1 | 3 | 3 | 44 |
| 14 | Man4 | 2 | 9 | 9 | 77 |
| 15 | Man4 | 3 | 27 | 25 | 97 |
| 4 | Man9-N3 | | 1 | 1 | — |
| 16 | Man9 | 1 | 3 | 3 | 73 |
| 17 | Man9 | 2 | 9 | 9 | 98 |
| 18 | Man9 | 3 | 27 | 25 | 98 |

Interaction of Oligomannose Dendrons with 2G12.

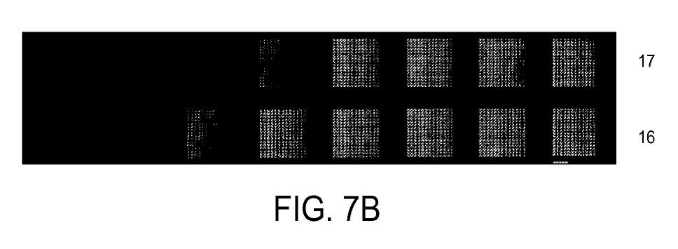

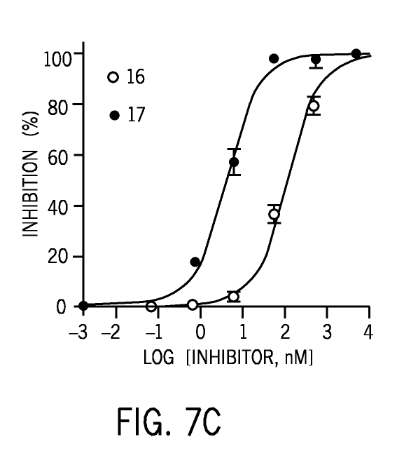

Binding of glycodendrons to 2G12 was compared with the corresponding monomers 3 and 4. In previous studies, Man₄ 1 was immobilized (FIG. 3B) onto an N-hydroxysuccinimide (NHS) activated glass slide (normal NHS density) to determine the surface dissociation constant (K_{D,surf}) of various proteins. In a modified assay, fluorescently labeled antibody and 2G12 were coincubated with oligomannosyl dendrons 13-18 (FIG. 3c) and competed for immobilized Man₄ on the same slide, as shown according to data illustrated in FIG. 7.

According to implementations of experimental data as shown in FIG. 7, measurements of oligomannose dendrons-2G12 complex interaction by glycan array competition assay are shown. In FIG. 7A, there is shown design of glycan array-based competition assay (not drawn to scale). In FIG. 7B, a representative microarray slide is shown, with compound 16 shown on the bottom row and compound 17 shown on the top row, which are used as inhibitors; concentrations from left to right are 50 M, 5 M, 500 nM, 50 nM, 5 nM, 500 pM, 50 pM, and 0. (Scale bar: 1 mm.) In FIG. 7C, inhibition curves of compounds 16 and 17 are shown for the determination of solution dissociation constant.

The IC₅₀ values of these glycodendrons are shown in Table 2.

TABLE 2
Oligomannose dendrons as inhibitors of 2G12 binding to multivalent glycan displays;
*Extrapolated from the concentration of 40% inhibition.

| | Glycan array | | Glycan array assay, high-density | | | | |
|---|---|---|---|---|---|---|---|
| | assay normal | | IC50,^{M} | | | 2G12/gp120 | |
| | Per | Per | Per | Per | | Per | Per |
| Compound | dendron | oligomannos | dendron | oligomannos | K_{D,sol,}^{M} | dendron | oligomann |
| 3 | 42 | 42 | 2,100 | 2,100 | 350 ± 98 | 1,100 | 1,100 |
| 13 | 0.29 | 0.87 | 100 | 300 | 17 ± 5.6 | 160 | 480 |
| 14 | 0.0046 | 0.041 | 1.2 | 11 | 0.21 ± 0.088 | 10* | 90* |
| 15 | 0.0041 | 0.10 | 0.022 | 0.56 | 0.0039 ± 0.0013 | 0.24 | 6.0 |
| 4 | 18 | 18 | 1,000 | 1,000 | 180 ± 99 | 530 | 530 |
| 16 | 0.095 | 0.29 | 3.5 | 11 | 0.61 ± 0.19 | 36 | 107 |
| 17 | 0.0030 | 0.027 | 0.020 | 0.18 | 0.0034 ± 0.00049 | 0.54 | 4.8 |
| 18 | 0.0031 | 0.078 | 0.018 | 0.46 | 0.0031 ± 0.00041 | 0.29 | 7.3 |

A strong increase in avidity to 2G12 was noted compared with monomeric oligomannose for the first (13 and 16) and second generations (14 and 17), whereas the third-generation glycodendron (15 and 18) remained at the same level as the second-generation glycodendron. To assess the validity of the new glycan microarray assay, a standard gp120/2G12 competition ELISA was performed, in which the analytes compete with surface-bound gp120 for uncomplexed 2G12. IC₅₀ in both assays improved rapidly with increasing generation for both Man₄ and Man₉ dendrons.

However, the glycan microarray cannot discriminate the efficacy of second- and third-generation Man₄ dendrons, which showed a 40-fold IC₅₀ difference in a gp120/2G12 ELISA. It is believed the incompatibility was due to the relatively low density of Man₄ on the microarray, which was not accurately mimicking the high-density glycan presentation on the surface of gp120. To correct, high-NHS-density slides were used for Man₄ immobilization. The resulting high-density Man₄ slide exhibited a matched trend in both assays (Table 2), suggesting that higher surface Man₄ density better mimics the surface of gp120. The solution dissociation constants (K_{D,sol}) for each glycodendron, as determined with high-density Man₄ slides, are reported in Table 2.

In both ELISA and high-density glycan array assays, the improvement of IC₅₀ for the dendrons relative to monomeric oligosaccharide is much greater than an additive effect, as shown in Table 2. All glycodendrons showed superior IC₅₀ per oligomannose unit relative to monomeric ligands, showing a synergistic multivalent effect.

ELISA results using dendrons 15, 17, and 18 showed improved inhibition compared with Man₉(GlcNAc)₂ clusters observed elsewhere. This may be partly due to the dendrons' higher valency or better presentation to the multiple-antibody glycanbinding sites on 2G12. It also has been demonstrated that a high-mannose dimer can be synthesized in a distance-fixed manner to give superior 2G12 affinity, but the internal flexibility, which should be minimized for an optimal synthetic vaccine, cannot be controlled in this case. Alternatively, the use of a rigid high-mannose cluster constrained the flexibility, but this control may decay rapidly when a longer linker is used to make a larger cluster. The use of glycodendrons may be a solution to this dilemma because the oligosaccharide valency increases as the structure grows larger, and therefore the internal flexibility is controlled to a certain level even within a large structure.

Based on the binding data in Table 2, the second-generation Man₉ dendron 17 is a promising candidate for vaccine development. It was chosen because it has a similar IC₅₀ to the third-generation dendrons (15 and 18) in disrupting the gp120-2G12 interaction but has a smaller size to facilitate synthesis and carrier protein conjugation.

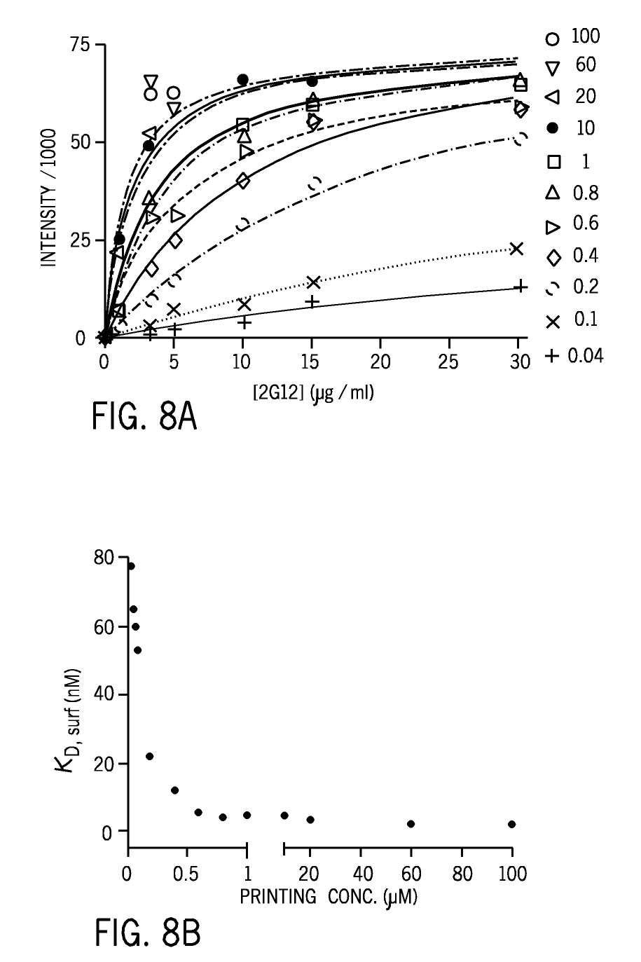

To further test its multivalent efficacy, 17 was immobilized onto a normal-NHS-density slide (FIG. 3d) at varying printing concentrations. The measured K_{D,surf} of 17 (3.5 nM) was significantly stronger than the K_{D,surf} of 2 (830 nM) on the same slide, indicating that the density of Man₉ on the dendron is higher than on the glass slide. The finding that the saturated K_{D,surf} of 17 is comparable to the K_{D,sol} for 17 (3.4 nM; FIG. 8 and Table 2) also suggested that the enhanced 2G12 complex avidity came from dendrimeric display of Man₉ rather than the pseudomultivalency arising from the close proximity of the surface-immobilized molecules. Moreover, the K_{D,surf} of 17 remains strong in the case of lower printing concentration, which is contrary to the results observed in Man₉ monomer, in which a high critical printing concentration was observed. It was reasoned that part of this phenomenon may arise from the high density of Man₉ on glycodendron 17, so that it does not require dense surface immobilization to achieve tight binding to 2G12. Overall, the glycodendron 17 appears to be an effective mimic of the gp120 surface, and it is suitable for conjugation to a carrier protein as a vaccine candidate. The critical printing concentration of 17 for 2G12 complex binding was found to be 400 nM (see FIG. 8), compared with 40 M for Man₄ on the same surface. The detection limit of this glycodendron slide for the 2G12 complex was 0.05 g/ml (Man₄ slide: 3 g/ml), which is low enough to be suitable for diagnostic use.

According to implementations of experimental data shown in FIG. 8, the properties of glycodendron 17-coated sides is shown. In FIG. 8A, representative binding curves of fluorescent 2G12 complex with glycodendron 17 arrayed on glass slide at different printing concentration, ranging from 40 nM to 100 M are shown. FIG. 8B illustrates a Calculated K_{D,surf} plot against 17 printing concentrations.

Interaction of Oligomannose Dendron with DC-SIGN.

The success in enhancing oligomannose-2G12 complex binding by dendrimeric scaffolds lead to testing of their affinity for DC-SIGN. Because the optimal size and the oligomannose density for 2G12 complex seem to be achieved at the second-generation Man₉ dendron 17, this construct was tested for DC-SIGN binding in a similar glycan microarray assay. Competitive binding was performed on the same high-density Man₄ slide, with the Fc-DC-SIGN fusion protein detected with Cy3-labeled anti-human IgG antibody. The Man₉ dendron 17 showed good competition against surface-bound Man₄ for Fc-DC-SIGN, whereas the Man₄ dendron 14 was weaker. Table 3 summarizes these results, which are consistent with a previous reports suggesting that the monomeric branched high mannose binds better than linear glycans.

TABLE 3
Oligomannose dendrons as inhibitors of Fc-DC-SIGN
binding to multivalent glycans

| | Glycan array assay | gp120/DC-SIGN-Fc |
|---|---|---|
| Compound | IC_{50,} μM | ELISA IC₅₀, μM |
| 14 | 0.16 | 0.020 |
| 17 | 0.026 | 0.008 |
| D-Mannose | — | 8,500 |

It was then determined whether oligomannose dendrons are able to interfere with the binding between gp120 and DC-SIGN, which is likely to be a key step for dendritic cell-mediated CD4 T cell HIV infection. Gp120/Fc-DC-SIGN ELISA was performed, in a similar setting as gp120/2G12 ELISA, to evaluate the inhibition activity of glycodendrons 14 and 17. Indeed, the second-generation glycodendrons demonstrated excellent inhibition activity in the nanomolar range, in contrast to the millimolar range from the reference mannose (see Table 3). In these experiments, no inhibition was observed for the unglycosylated alkynyl dendron 9 (up to 0.1 mM), showing that the multivalent oligomannose is responsible for DC-SIGN binding.

It was thereafter determined whether these glycodendrons bind DC-SIGN presented on cell surface. We synthesized fluorescence-labeled dendrons 14 and 17 by connecting fluorescein to the amine group via an oligo(ethylene glycol) linker and used flow cytometry to monitor their interaction to cell-surface DC-SIGN.

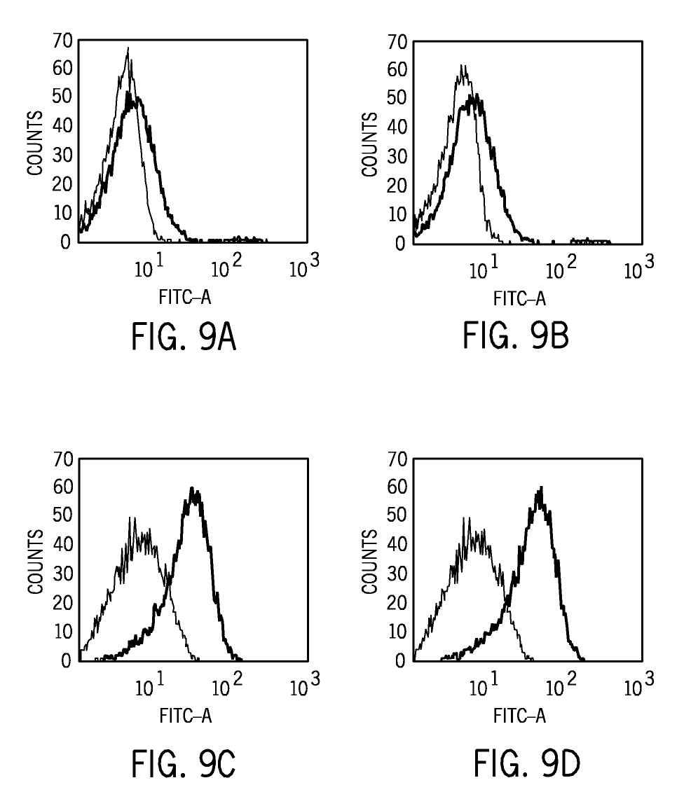

As shown according to implementations illustrated by the experimental data of FIG. 9, both glycodendrons stain DC-SIGN-expressing Jurkat cells with a stronger fluorescent intensity compared with the negative control Jurkat cells. The results indicate that the oligomannose dendrons interact with DC-SIGN on cell surface. In the same setting, we found that immature monocyte-derived dendritic cells (MDDC) were intensely labeled by these fluorescent dendrons, as shown in FIG. 9. Because DC-SIGN is not the only mannose-binding lectin on MDDC, it is possible that the multivalent high-mannose glycans also bind to other mannose-binding proteins, which may also contribute to viral transmission.

According to implementations of experimental data shown in FIG. 9, flow cytometry histograms showing fluorescein-labeled glycodendrons 14 (green in FIGS. 9A and 9C) and 17 (blue in FIGS. 9B and 9D) binds DC-SIGN-expressing Jurkat cells (FIGS. 9A and 9B) or MDDCs (FIGS. 9C and 9D). Mock-transfected Jurkat cells stained with the same conditions are shown in red in FIGS. 9A and 9B. The fluorescent levels of mock-transfection control are the same as unstained cells. Unstained MDDCs serve as the negative control (red) for glycodendron-stained MDDCs in FIGS. 9C and 9D.

Sexual transmission is a major route for HIV infection, in which the dendritic cells enhance the infection of CD4 T cells. Therefore, inhibiting the gp120-DC-SIGN interaction, which is likely the key step of HIV-dendritic cells binding, has become a strategy for preventing infection. Our glycodendrons inhibit the DC-SIGN-gp120 interaction, demonstrating their potential as antiviral agent for preventing sexual transmission of HIV-1. Furthermore, as well defined structures, our glycodendrons may be useful for investigating the “macro” structure requirement of ligands for DC-SIGN or other receptors.

Thus, the present inventors have developed a strategy for the efficient syntheses of oligomannose dendrons, in which the high-density oligomannose mimics the glycans on the surface of HIV-1 and the monomeric glycan immobilized on glass slides. The binding properties of these glycodendrons were characterized by glycan microarray assay. The inhibition of glycodendrons on gp120 interacting with 2G12 and DC-SIGN demonstrated that these glycodendrons, especially the second-generation Man₉ dendron, have the potential for use in the development of both carbohydrate vaccine candidates and antiviral agents. HIV uses its glycan shield to evade the immune response, but the unusual high glycan density and the existence of conserved oligomannosides, evidenced by the discovery of the broadly neutralizing antibody 2G12, suggest that targeting of these carbohydrates may be a promising approach. From this point of view, multivalent display of carbohydrates that have higher binding affinity/avidity may be a practical solution for inducing 2G12-like antibodies and blocking mannose-binding-protein-mediated viral infection.

### Pharmaceutical Compositions

The instant disclosure also provides pharmaceutical compositions. In some implementations, the pharmaceutical compositions comprise the oligomannose dendrimers of the present disclosure. In such pharmaceutical compositions, the oligomannose dendrimer form the “active compound.” According to implementations, the pharmaceutical compositions are administered to a subject to innoculate the subject against HIV infection by causing the subject to form antibodies. According to other implementations, the pharmaceutical compositions are administered to a subject infected with HIV to compete with virus binding to immune cells and thereby inhibit binding of HIV viruses to dendridic cell-surface DC-SIGN or other mannose-binding proteins to prevent dendritic cell-enhanced CD4⁺ T cell infection.

In addition to active compound, the pharmaceutical compositions preferably comprise at least one pharmaceutically acceptable carrier. As used herein the language “pharmaceutically acceptable carrier” includes solvents, dispersion media, coatings, antibacterial and antifungal agents, isotonic and absorption delaying agents, and the like, compatible with pharmaceutical administration. Supplementary active compounds can also be incorporated into the compositions. A pharmaceutical composition is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Subject as used herein refers to humans and non-human primates (e.g., guerilla, macaque, marmoset), livestock animals (e.g., sheep, cow, horse, donkey, pig), companion animals (e.g., dog, cat), laboratory test animals (e.g., mouse, rabbit, rat, guinea pig, hamster), captive wild animals (e.g., fox, deer) and any other organisms who can benefit from the agents of the present disclosure. There is no limitation on the type of animal that could benefit from the presently described agents. Human subjects are expressly contemplated. A subject regardless of whether it is a human or non-human organism may be referred to as a patient, individual, animal, host, or recipient.

Pharmaceutical compositions suitable for injectable use include sterile aqueous solutions (where water-soluble) or dispersions and sterile powders for the extemporaneous preparation of sterile injectable solutions or dispersion. For intravenous administration, suitable carriers include physiological saline, bacteriostatic water, Cremophor EL.™ (BASF, Parsippany, N.J.) or phosphate buffered saline (PBS). In all cases, the composition should be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, or sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from a container or dispenser which contains a suitable propellant, e.g., a gas such as carbon dioxide, or a nebulizer. Other delivery methods and devices common in the art, including mechanically actuated atomizing-like devices are expressly contemplated.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one implementation, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to cell-specific antigens) can also be used as pharmaceutically acceptable carriers. These can be prepared according to methods known to those skilled in the art, for example, as described in U.S. Pat. No. 4,522,811, incorporated by reference herein.

It is advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier.

Toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds which exhibit high therapeutic indices are preferred. While compounds that exhibit toxic side effects can be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in subjects. The dosage of such compounds lies preferably within a range of circulating concentrations that include the ED₅₀ with little or no toxicity. The dosage can vary within this range depending upon the dosage form employed and the route of administration utilized. For any compound used in the method of the disclosure, the therapeutically effective dose can be estimated initially from cell culture assays. A dose can be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in subjects. Levels in plasma can be measured, for example, by high performance liquid chromatography.

As defined herein, a therapeutically effective amount of an active compound of the disclosure may range, for examples, from about 0.001 to 30 mg/kg body weight, about 0.01 to 25 mg/kg body weight, about 0.1 to 20 mg/kg body weight, or about 1 to 10 mg/kg, 2 to 9 mg/kg, 3 to 8 mg/kg, 4 to 7 mg/kg, or 5 to 6 mg/kg body weight. Without limitation, the active compound can be administered between one time per week and three or more times per day, for between about 1 to 10 weeks, for example between 2 to 8 weeks, between about 3 to 7 weeks, or for about 4, 5, or 6 weeks. The skilled artisan will appreciate that certain factors can influence the dosage and timing required to effectively treat a subject, including but not limited to the severity of the disease or disorder, previous treatments, the general health or age of the subject, and other diseases present. Moreover, treatment of a subject with a therapeutically effective amount of a pharmaceutical composition of the disclosure can include a single treatment or, preferably, can include a series of treatments.

### EXAMPLES

### Example 1

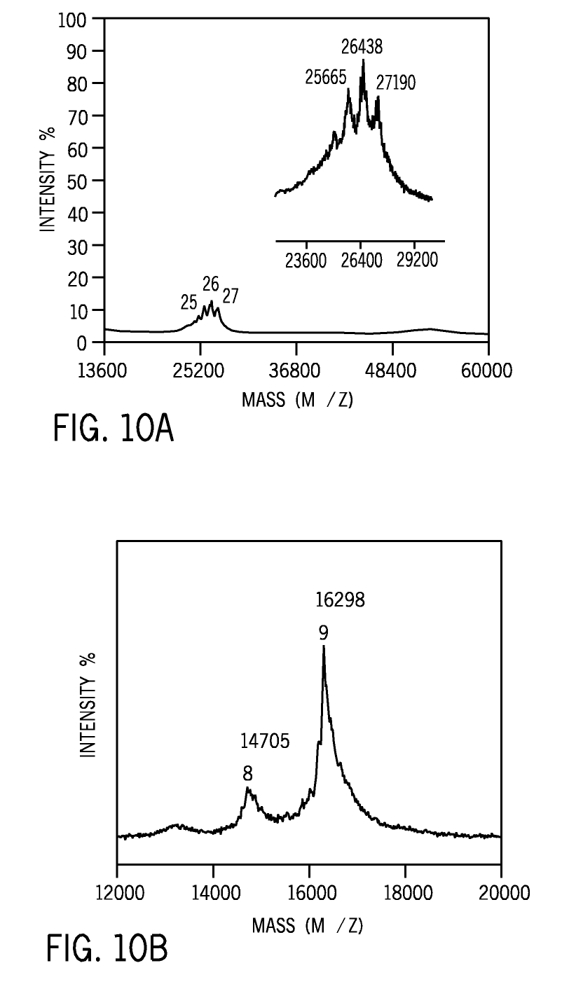

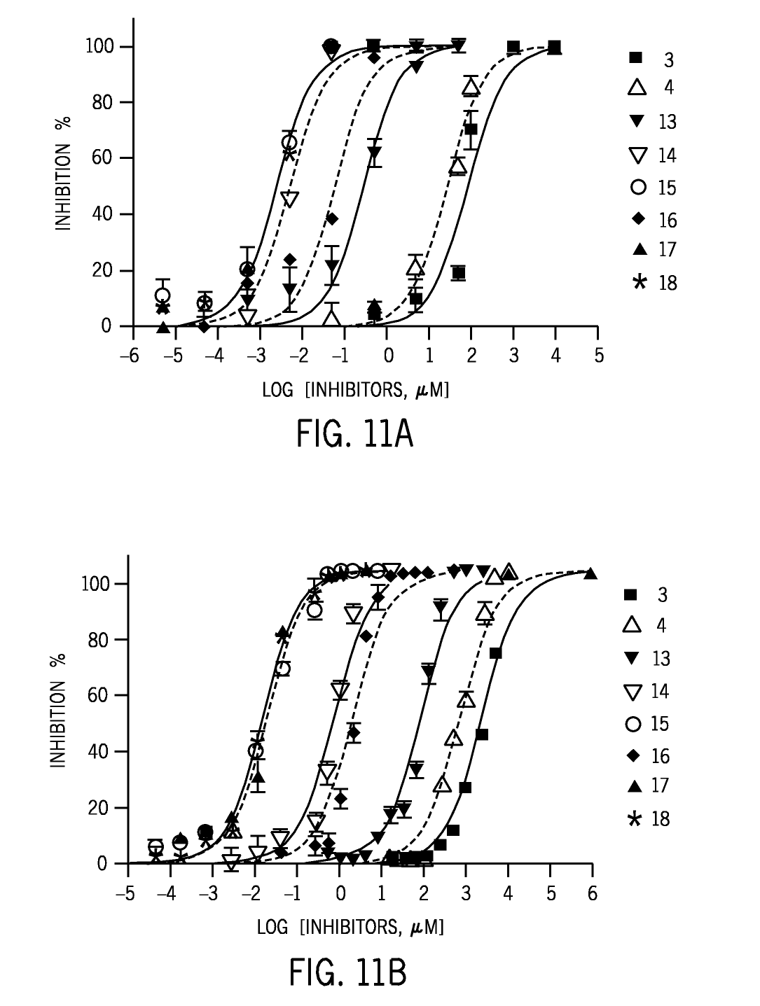

IC50 Determined by the Microarray Competitor Assay for 2G1 2 Complex. Serial diluted competitors (1.5 I) were mixed with 1.5 I of 50 g/ml (based on 2G12, for high-density Man₄ slide: 15 g/ml) 2G12-Cy3-labeled goat antihuman IgG complex (44). The 3-I mixtures in PBS-BT buffer (1% BSA and 0.05% Tween 20 in PBS) were applied directly to each subarray. After incubation in a humidified chamber for 1 h, the slide was rinsed sequentially with PBS, PBS-T buffer (0.05% Tween 20 in PBS), and distilled water, and then centrifuged at 200×g for 5 min to ensure a complete dryness. The array was then imaged at 5-Å resolution with an A595 laser on an ArrayWorx microarray reader (Applied Precision) to measure the fluorescence. ArrayVision 8.0 was used for the fluorescence analysis and extraction of data (Applied Precision). Binding curves are shown in FIGS. 10 and 11

### Example 2

Competition ELISA. Microtiter plate wells (flat-bottom; Costar type 3690 from Corning) were coated with 50 ng per well gp120 JR-FL overnight at 4° C. in PBS. All subsequent steps were performed at room temperature. The wells were then washed four times with PBS/0.05% (vol/vol) Tween 20 (Sigma) before blocking for 1 h with 3% (mass/vol) BSA. IgG 2G 12, diluted to 0.5 g/ml (25 ng per well) with 1% (mass/vol) BSA/0.02% (vol/vol) Tween 20/PBS (PBS-BT), was then added for 2 h to the antigen-coated wells in the presence of serially diluted oligomannoses or glycodendrons. Unbound Abwas removed by washing four times as described above. Bound 2G1 2 was detected with an alkaline phosphatase-conjugated goat anti-human IgG F(ab′)₂ Ab (Pierce) diluted 1:1,000 in PBS-BT. After 1 h, the wells were washed four times, and bound Ab was visualized with p-nitrophenyl phosphate substrate (Sigma) and monitored at 405 nm.

### Example 3

### Syntheses of Compounds

General. All chemicals were purchased from Aldrich or Acros and used without further purification. Reactions were monitored with analytical thin layer chromatography (TLC) in EM silica gel 60 F254 plates and visualized under UV (254 nm) or staining with acidic cerium ammonium molybdate or ninhydrin. Flash column chromatography was performed on silica gel 60 (35-75 μm, EM Science) or latrobeads 6RS-8060 (Mitsubishi Kagaku Iatron Inc). ¹H-NMR and ¹³C NMR spectra were recorded on a Bruker DRX-500 spectrometer at 20° C. ¹H NMR spectra are reported in this order: chemical shift; multiplicity; coupling constant(s); number(s) of proton. The MALDI-TOF mass spectrometry was performed on a PerSeptive Biosystems Voyager-DE Biospectrometry workstation using 2,5-dihydroxybenzoic acid (DHB) as the matrix. The data were analyzed by Data Explorer software v. 3.2.

Boc-G1-alkyne, 7. To a stirred solution of tri-acid compound 6 (0.47 g, 0.93 mmol), propargylamine (0.26 g, 4.66 mmol), HOBt (0.57 g, 3.73 mmol), and DIEA (0.81 ml, 4.66 mmol) in DMF (10 ml) at 0° C. was added EDC (0.89 g, 4.66 mmol). The reaction mixture was allowed to return to room temperature gradually and stirred overnight, which was followed by evaporating in vacuo and column chromatography (MeOH:CHCl₃=1:20) to give purified product 7 (0.43 g, 75%) as colorless oil.

¹H NMR (500 MHz, CDCl₃) δ1.44 (s, 9H), 2.27 (t, J=2.4 Hz, 3H), 2.58-2.40 (m, 8H), 3.39 (dd, J₁=12.1 Hz, J₂=5.9 Hz, 2H), 3.69 (s, 6H), 3.73 (t, J=5.7 Hz, 6H), 4.06 (dd, J₁=5.5 Hz, J₂=2.6 Hz, 6H), 5.30 (s, 1H), 6.33 (s, 1H), 7.00 (s, 3H); ¹³C NMR (125 MHz, CDCl₃) δ28.28, 28.79, 36.15, 36.83, 37.03, 59.64, 67.08, 69.28, 71.19, 79.17, 79.95, 155.96, 171.28, 172.04; ESI-TOF HRMS calculated for C₃₀H₄₆N₅O₉ (M+H)⁺: 620.3290, found 620.3290.

NH₂-G1-alkyne, 8. To a flask containing compound 7 (0.14 g, 0.22 mmol) in CH₂Cl₂ (1 ml) was added TFA solution (1 ml, 50% v/v in CH₂Cl₂) at 0° C. After all starting material were consumed, the reaction mixture was evaporated in vacuo. The residual TFA in the mixture was removed by Amberlite IRA-743 ion-exchange resin. The crude product (0.11 g, 96%) was used in the next step without further purification.

¹H NMR (500 MHz, CD₃OD) δ2.43 (t, J=5.9 Hz, 6H), 2.58 (t, J=2.4 Hz, 3H), 2.62 (t, J=6.4 Hz, 2H), 3.16 (t, J=6.1 Hz, 2H), 3.60-3.75 (m, 12H), 3.96 (d, J=2.2 Hz, 6H); ¹³C NMR (125 MHz, CD₃OD) δ29.42, 33.32, 37.15, 61.56, 68.35, 69.87, 72.27, 80.75, 172.18, 173.61; ESI-TOF HRMS calculated for C₂₅H₃₈N₅O₇ (M+H)⁺: 520.2766, found 520.2779.

Boc-G2-alkyne, 9. To a stirred solution of tri-acid compound 6 (0.022 g, 0.043 mmol), compound 8 (0.092 g, 0.176 mmol), HOBt (0.02 g, 0.132 mmol), and DIEA (0.03 ml, 0.176 mmol) in DMF (1 ml) at 0° C. was added EDC (0.042 g, 0.22 mmol). The reaction mixture was allowed to return to room temperature gradually and stirred 24 h, which was followed by evaporating in vacuo and column chromatography (MeOH:CHCl₃=1:5) to give purified product 9 (0.084 g, 97%) as colorless gum.

¹H NMR (500 MHz, CD₃OD) δ 1.51 (s, 9H), 2.44-2.62 (m, 32H), 2.70 (t, J=2.6 Hz, 9H), 3.36 (t, J=6.6 Hz, 2H), 3.50 (t, J=7.0 Hz, 6H), 3.72-3.87 (m, 48H), 4.06 (d, J=2.2 Hz, 18H); ¹³C NMR (125 MHz, CD₃OD) δ28.88, 29.49, 37.21, 37.32, 37.58, 37.86, 38.25, 47.88, 61.49, 61.58, 68.47, 68.71, 70.04, 72.44, 80.15, 80.90, 158.16, 173.59, 173.68, 173.85, 173.96; ESI-TOF HRMS calculated for C₉₆H₁₄₂N₁₇O₃₀ (M+H)⁺: 2013.0103, found 2013.0113.

NH₂-G2-alkyne, 10. The same procedure as for compound 8, (99%).

¹H NMR (500 MHz, CD₃OD) δ2.50-2.35 (m, 32H), 2.62 (t, J=2.4 Hz, 9H), 3.15-3.24 (m, 2H), 3.41 (t, J=7.0 Hz, 6H), 3.63-3.78 (m, 48H), 3.98 (d, J=2.6 Hz, 18H); ¹³C NMR (125 MHz, CD₃OD) δ 29.47, 37.17, 37.26, 37.44, 61.47, 61.58, 68.44, 68.64, 69.99, 72.44, 80.89, 172.33, 173.57, 173.64, 173.84; ESI-TOF HRMS calculated for C₉₁H₁₃₄N₁₇O₂₈ (M+H)⁺: 1912.9584, found 1912.9564.

Boc-G3-alkyne, 11. To a stirred solution of tri-acid compound 6 (5.9 mg, 0.012 mmol), compound 10 (0.079 g, 0.041 mmol), HOBt (8.0 mg, 0.051 mmol), and DIEA (0.013 ml, 0.072 mmol) in DMF (0.8 ml) at 0° C. was added EDC (0.014 g, 0.072 mmol). The reaction mixture was allowed to return to room temperature gradually and stirred 72 h, which was evaporated in vacuo. The residue was purified by silica gel column chromatography (MeOH:CHCl₃=1:2) and followed by another size-exclusion column (Bio-gel P-10, Bio-Rad) to give purified product 11 (0.0285 g, 40%) as colorless gum.

¹H NMR (500 MHz, CD₃OD) δ 1.52 (s, 9H), 2.44-2.56 (m, 104H), 2.72 (t, J=2.6 Hz, 27H), 3.50 (t, J=7.0 Hz, 26H), 3.72-3.85 (m, 156H), 4.07 (d, J=2.6 Hz, 54H); ¹³C NMR (125 MHz, CD₃OD) δ 29.01, 29.54, 37.25, 37.35, 37.62, 61.52, 61.54, 68.52, 68.76, 70.08, 72.53, 80.99, 81.01, 173.60, 173.63, 173.67, 173.80; MALDI-TOF calculated for C₂₉₄H₄₃₀N₅₃O₉₃ (M+H)⁺: 6191, found 6192.

### Example 4

Representative procedures for conjugating oligomannose to alkynyl dendron via CuAAC reaction (Boc-Gn-Alkyne→Boc-Gn-Manₓ), 13˜18. To a stirred solution of Man₉-N₃ (5.0 mg, 3.14 μmol) and Boc-G3-Alkyne (0.57 mg, 0.09 μmol) in 0.2 ml H₂O was added aqueous CuSO₄ (20 mM, 10 μl), triazole ligand 12 (20 mM in DMSO, 10 μl) and sodium ascorbate (20 mM, 20 μl). The mixture was stirred for 2 hr, and analyzed by MALDI-TOF MS to confirm the completeness of the reaction. The mixture was then repeatedly centrifugal filtered (Millipore Microcon YM-3) and washed to give white solid as product 18 (4.3 mg, 98%) after concentrated.

Boc-G1-Man₄, 13. ¹H NMR (500 MHz, D₂O) δ1.13-1.27 (m, 15H), 1.41-1.52 (m, 6H), 1.72-1.80 (m, 6H), 2.18 (t, J=5.9 Hz, 2H), 2.35 (t, J=5.7 Hz, 6H), 3.10 (t, J=6.3 Hz, 2H), 3.33-3.92 (m, 90H), 4.26 (t, J=7.0 Hz, 6H), 4.32 (s, 6H), 4.66 (s, 3H), 4.91 (s, 3H), 5.16 (s, 3H), 5.21 (s, 3H), 7.77 (s, 3H).

MALDI-TOF calculated for C₁₁₇H₁₉₈N₁₄O₇₂Na (M+Na)⁺: 2974, found 2975.

Boc-G2-Man₄, 14. ¹H NMR (500 MHz, D₂O) δ1.14-1.28 (m, 27H), 1.46-1.54 (m, 18H), 1.66-1.82 (m, 18H), 2.27-2.32 (m, 8H), 2.34-2.41 (m, 24H), 3.13-3.17 (m, 2H), 3.25-3.30 (m, 6H), 3.37-4.02 (m, 282H), 4.28 (t, J=6.8 Hz, 18H), 4.35 (s, 18H), 4.70 (s, 9H), 4.95 (s, 9H), 5.20 (s, 9H), 5.25 (s, 9H), 7.81 (s, 9H).

MALDI-TOF calculated for C₃₅₇H₆₀₀N₄₄O₂₁₉Na (M+Na)⁺: 9031, found 9028.

Boc-G3-Man₄, 15. MALDI-TOF calculated for full conjugation C₁₀₇₇H₁₈₀₇N₁₃₄O₆₆₀ (M+H)⁺: 27178. FIG. 10 illustrates the full spectrum.

Boc-G1-Man₉, 16. ¹H NMR (500 MHz, D₂O) δ1.15-1.25 (m, 15H), 1.44-1.52 (m, 6H), 1.73-1.80 (m, 6H), 2.18 (t, J=5.9 Hz, 2H), 2.36 (t, J=5.3 Hz, 6H), 3.10 (t, J=5.9 Hz, 2H), 3.32-3.99 (m, 180H), 4.26 (t, J=6.8 Hz, 6H), 4.32 (s, 6H), 4.71 (s, 3H), 4.91 (s, 9H), 5.01 (s, 3H), 5.17 (s, 3H), 5.20 (s, 3H), 5.26 (s, 3H), 7.77 (s, 3H). MALDI-TOF calculated for C₂₀₇H₃₄₈N₁₄O₁₄₇Na (M+Na)⁺: 5405, found 5405.

Boc-G2-Man₉, 17. MALDI-TOF calculated for full conjugation C₆₂₇H₁₀₅₁N₄₄O₄₄₄ (M+H)⁺: 16301, found 16298. Also see Figure S2 for the full spectrum.

Boc-G3-Man₉, 18. MALDI-TOF calculated for full conjugation C₁₈₈₇H₃₁₅₇N₁₃₄O₁₃₃₅ (M+H)⁺: 49055. FIG. 6 illustrates the full spectrum.

### Example 5

### Microarray Experiments, Microarray Fabrication

Man₄ on normal NHS density slide. NHS-coated glass slides (slide H, Schott North American) were printed by robotic pin deposition of ˜0.7 nl of Man₄, 1, with concentrations of 100, 75, 50, 40, 30, 20, 15, 10, 7.5, 5, 3, 1, 0.75, 0.5, 0.25, 0.1 μM in print buffer (300 mM phosphate, pH 8.5 containing 0.005% Tween-20) from left to right with 16 replicates vertically placed in each sub-array and there were totally 16 replicates of subarrays on one slide. After the solvent of the printed matrix evaporated, the slide was washed with PBST (0.05% Tween 20) buffer and then treated with blocking solution (superblock blocking buffer in PBS, Pierce) at room temperature for 1 h. The slides were then washed with PBS buffer, dried, and stored in dessicator.

Man₄ on high NHS density slide: Similar procedure as for normal density slides was used. The NHS activated slides were manufactured by GE Healthcare (CodeLink HD). The printing concentration for Man₄, 1, was 100, 80 and 60 μM.

Second generation Man₉ dendron 17 on normal NHS density slide: Similar procedure as for normal density Man₄ slide was used. Derivative of 17 with a linker connecting free amine was used for printing. The printing concentrations are 100, 80, 60, 40, 20, 10, 8, 6, 4, 2, 1, 0.8, 0.6, 0.4, 0.2, 0.1, 0.08, 0.06, 0.04, and 0.02 μM. The K_{D,surf} of 2G12 complex was obtained (see Table 4) as previously described. The limit of detection is defined as the lowest 2G12 concentration applied to slide that resulted in signal to noise ratio greater than 10.

### Example 6

Determination of K_{D,surf} of glycodendron 17. The procedure is the same as that disclosed in Liang et al., “Quantitative analysis of carbohydrate-protein interactions using glycan microarrays: determination of surface and solution dissociation constants.” J Am Chem Soc 129:11177-11184 (2007), which is hereby incorporated by reference as if fully disclosed herein.

TABLE 4
Printed concentration for glycodendron 17 derivative and the
corresponding dissociation constants on the surface.

| | Printing conc. (μM) | | | | | | | | | |
|---|---|---|---|---|---|---|---|---|---|---|
| | 100 | 80 | 60 | 40 | 20 | 10 | 8 | 6 | 4 | 2 |
| K_{D,surf} (nM) | 2.2 | 1.8 | 2.1 | 2.1 | 3.4 | 4.5 | 3.8 | 3.9 | 3.6 | 4.2 |
| | Printing conc. (μM) | | | | | | | | | |
| | 1 | 0.8 | 0.6 | 0.4 | 0.2 | 0.1 | 0.08 | 0.06 | 0.04 | 0.02 |
| K_{D,surf} (nM) | 4.5 | 4.0 | 5.3 | 12 | 22 | 53 | 60 | 65 | 78 | 78 |

### Example 7

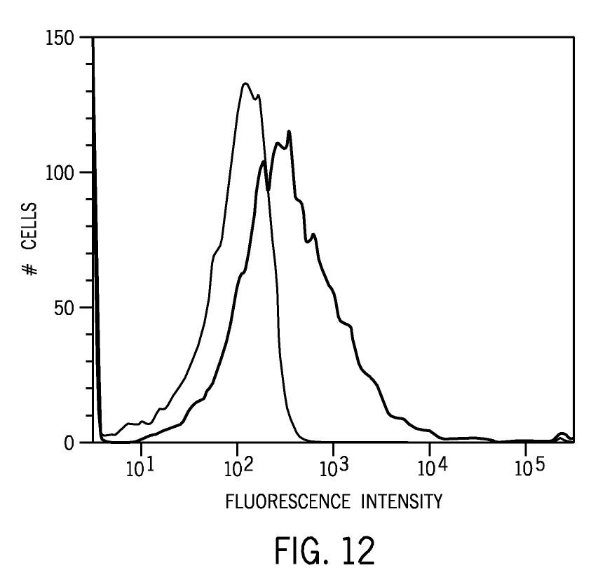

Microarray competitor assay for 2G12 complex. Results for a microarray competitor assay are shown in FIGS. 12 and 13, and performed as well known and understood by a person of ordinary skill in the art.

### Example 8

K_{D,sol} determination. The solution equilibrium dissociation constant (K_{D,sol}) for oligomannose-2G12 complex interactions can be determined using microarrays in a competitive assay. The equation that describes the binding of the two ligands to the same site on the protein is identical to that for the competitive inhibition of an enzyme-catalyzed reaction. It is possible to take advantage of the convenience of IC₅₀ measurements and still report inhibitory potency in terms of true K_{D,sol} values (Table S1). The final forms of the relationship can be simply presented as:

$${K}_{D , sol} = \frac{{IC}_{50}}{1 + \frac{[ Po ]}{{K}_{D , surf}}} \qquad ( 1 )$$

The limit of detection is defined as the lowest 2G12 concentration applied to slide that resulted in signal to noise ratio greater than 10. For the 17 immobilized slide, the limit was found at 0.05 μg/ml, where for the normal Man₄ slide is 3 μg/ml.

### Example 9

IC₅₀ determined by the microarray competitor assay for Fc-DC-SIGN. 1.5 μl of serial diluted of competitors were mixed with 1.5 μl 80 μg/ml Fc-DC-SIGN, under buffered condition (binding buffer: 2 mM CaCl₂, 2 mM MgCl₂, 150 mM NaCl, 0.05% Tween20, 1% BSA, 20 mM Tris-HCI pH 7.4). The mixtures (3 μl) were applied directly to each sub-array. After incubation in a humidified chamber for 1 h at room temperature, it was rinsed sequentially with PBS, PBS-T buffer (0.05% Tween 20 in PBS) and distilled water. After residual water removed, 15 μl of Cy3 labeled goat anti-human IgG antibody (0.01 μg/ml in binding buffer) was applied to each sub-matrix and incubated in moisture chamber for 30 min. The following washing and analyzing process was as described for 2G12 complex experiments.

### Example 10

Competition ELISA for Fc-DC-SIGN. DC-SIGN competitions were done with 1.5 μg/ml Fc-DC-SIGN as described above with a few modifications since DC-SIGN is a C-type lectin that requires Ca²⁺ for binding. A Tris (10 mM, pH7.8) buffer containing NaCl (150 mM), i.e., TBS, replaced PBS in the abovementioned buffers used for coating, blocking and washing. After the initial wash step, CaCl₂ (10 mM) was included in the blocking solution and all subsequent incubation and wash buffers until substrate addition. DC-SIGN-Fc binding was detected with an alkaline phosphatase-conjugated goat anti-human IgG, Fcγ-specific, Ab (Jackson ImmunoResearch Laboratories, Inc.) diluted 1:1000.

### Example 11

Over-expression of cell surface DC-SIGN. Full length cDNA encoding human DC-SIGN was PCR-amplified and subcloned into pFLAG-CMV1. The construct pFLAG-CMV1-DC-SIGN was transfected to Jurkat cells by a MicroPorator (NanoEnTek, Seoul, Korea) according to the instructions. Briefly, Jurkat cells (2×10⁶) were mixed with 10 μg plasmid in 100 μl resuspension buffer, and were electroporated once with a pulse voltage of 1410 V and a pulse width of 30 ms. Cells were cultured in complete growth medium with no antibiotics for two days before surface staining.

### Example 12

Culture of monocyte-derived dendritic cells (MDDCs). Peripheral blood mononuclear cells (PBMCs) were isolated from white blood cell concentrates (obtained from San Diego Blood Bank, CA) by standard density gradient centrifugation with Ficoll-Paque (GE Healthcare). Monocytes were then purified with anti-CD14 microbeads (Miltenyi Biotec, Auburn, Calif.) and cultivated in RPMI 1640 medium (Invitrogen) supplemented with 10% FBS (Invitrogen), 800 U/ml human GM-CSF (Peprotech, Rocky Hill, N.J.) and 500 U/ml human IL-4 (Peprotech) for 6 days to differentiate to MDDCs.

### Example 13

Flow cytometric analysis. Cells were stained with Fluorescein-conjugated glycodendrons (50 pmol for 3×10⁵ cells) or mouse anti-DC-SIGN MAb (clone 120507; R&D Systems, Minneapolis, Minn.) in FACS buffer (1% FBS and 0.1% NaN₃ in PBS) at 4° C. for 20 min. Phycoerythrin (PE)-conjugated goat anti-mouse IgG was used to stain DC-SIGN subsequently at 4° C. for 20 min. Fluorescence intensity was analyzed by a LSR II™ (BD Biosciences, San Jose, Calif.) and CellQuest Pro (BD Biosciences).

As illustrated in FIG. 14, Jurkat cells were subsequently stained with anti-DC-SIGN and fluorescent secondary antibodies. Red, mock-transfected Jurkat cells. Green, pFLAG-CMV1-DC-SIGN-transfected Jurkat cells.

While the apparatus and method have been described in terms of what are presently considered to be the most practical and preferred implementations, it is to be understood that the disclosure need not be limited to the disclosed implementations. It is intended to cover various modifications and similar arrangements included within the spirit and scope of the claims, the scope of which should be accorded the broadest interpretation so as to encompass all such modifications and similar structures. The present disclosure includes any and all implementations of the following claims.

1. A method comprising:
addressing the infection of a human immunodeficiency virus (HIV) by administering a composition comprising oligomannose dendrons to a patient to induce production of antibodies that will recognize the HIV or compete with the HIV for DC-SIGN binding.

2. The method of claim 1, wherein the oligomannose dendrons comprise at least Man₄ molecules.

3. The method of claim 1, wherein the oligomannose dendrons comprise at least Man₉ molecules.

4. The method of claim 1, wherein the composition is administered to the patient to vaccinate the patient against HIV infection.

5. A composition comprising:
a oligomannose dendrimer and a pharmaceutically acceptable carrier.

6. The composition of claim 5, wherein the oligomannose comprises Man₄.

7. The composition of claim 5, wherein the oligomannose comprises Man₉.

8. The composition of claim 5, wherein the dendrimer is a second or third generation dendrimer.

9. A composition comprising:
a vaccine to address a human immunodeficiency virus infection comprising at least an oligomannose dendrimer.

10. The composition of claim 9, wherein the oligomannose comprises Man₄.

11. The composition of claim 9, wherein the oligomannose comprises Man₉.

12. The composition of claim 9, wherein the dendrimer is a second or third generation dendrimer.

13. A method comprising:
administering a composition to a patient at risk for acquiring a human immunodeficiency virus infection, the composition comprising an oligomannose dendrimer and a pharmaceutically acceptable carrier.

14. The composition of claim 14, wherein the oligomannose comprises Man₄.

15. The composition of claim 14, wherein the oligomannose comprises Man₉.

16. The composition of claim 14, wherein the dendrimer is a second or third generation dendrimer.

17. A method comprising:
administering a composition to a subject that is infected with a human immunodeficiency virus, the composition comprising an oligomannose dendrimer and a pharmaceutically acceptable carrier.

18. The composition of claim 17, wherein the oligomannose comprises Man₄.

19. The composition of claim 17, wherein the oligomannose comprises Man₉.

20. The composition of claim 17, wherein the dendrimer is a second or third generation dendrimer.

21. A method comprising:
manufacturing an oligomannose dendrimer have the steps shown in at least one of FIG. 4 and FIG. 5.

22. A product by the process of claim 21.

23. A method comprising:
screening at least one antibody for activity for efficacy against a human immunodeficiency virus by:
contacting the at least one antibody with a substrate having bound thereto oligomannose dendrimers, and
detecting the presence or absence of a probe.
